(19) United States
(12) Patent Application Publication
(10) Pub. No.: US 2021/0262021 A1
(43) Pub. Date: Aug. 26, 2021

(54) CLEAVABLE CO-OPERATIVE PRIMERS AND METHOD OF AMPLIFYING NUCLEIC ACID SEQUENCES USING SAME

(71) Applicant: ADVANCED THERANOSTICS INC., Hamilton (CA)

(72) Inventors: James Benson Mahony, Hamilton (CA); Syliva Chong, Hamilton (CA); David Charles Bulir, Hamilton (CA)

(21) Appl. No.: 16/972,738

(22) PCT Filed: Jun. 7, 2019
(86) PCT No.: PCT/CA2019/050807

§ 371 Date: Dec. 7, 2020

Related U.S. Application Data
(60) Provisional application No. 62/682,548, filed on Jun. 8, 2018.

(51) Int. Cl. C12Q 1/6853; C12Q 1/6876

(52) U.S. Cl. CPC C12Q 1/6853; C12Q 1/6876

(57) ABSTRACT

The present invention relates to an improved method for amplifying nucleic acid sequences using cleavable co-operative primers having a ribose base cleavage site, and a temperature stable polymerase enzyme.

### RELATED APPLICATIONS

This application claims priority from U.S. provisional application No. 62/682,548, filed on Jun. 8, 2018, the entire content of which is incorporated herein by reference.

### FIELD OF THE INVENTION

The invention relates to isothermal amplification and detection of DNA or RNA sequences, and in particular to isothermal amplification and detection using co-operative primers.

### BACKGROUND OF THE INVENTION

Nucleic acid amplification tests (NAATs) have become the cornerstone for microbiology laboratories, providing a same day diagnosis for a wide range of infections. Although polymerase chain reaction (PCR) has served laboratories well since its inception, PCR tests have significant disadvantages as they are labor intensive and relatively slow compared with newer isothermal amplification methods. Following the introduction of the first isothermal amplification methods (strand displacement amplification and loop-mediated isothermal amplification), several other methods have been introduced, and some of these can yield positive results in as little as 5-10 minutes. Point-of-care (POC) tests that are being designed to provide rapid and actionable results for healthcare providers at the time and place when patients first encounter the health care system require more rapid NAATs.

Traditional diagnostic testing for bacterial and viral infections involved virus isolation in cell culture, ELISA, serology, direct fluorescent antigen (DFA) staining of specimens and shell vial culture (SVC) using a panel of monoclonal antibodies. In the early 1990s the use of specific monoclonal antibodies raised against respiratory viruses allowed for the detection of these viruses within 3 hours using DFA staining or within 1-2 days using SVC for slowly growing viruses. This was far superior to the 8-10 days required for cell culture. Rapid EIA tests developed in the 1980s and 1990s for point-of-care testing for bacteria and viruses lacked sensitivity; the clinical sensitivities of these tests ranged from 20 to 90%, varying widely with the patient population being tested. These rapid EIA tests are therefore not recommended for use in critical care settings due to their low sensitivities.

### SUMMARY OF THE INVENTION

Improved methods and compositions are provided herein for performing strand displacement amplification that utilizes co-operative primers which contain an RNase H cleavage site.

In one aspect, there is provided a target-specific co-operative primer for amplifying a target polynucleotide region of a nucleic acid molecule, the primer comprising:
a 3′ to 5′ bumper sequence segment, and
a 5′ to 3′ inner primer sequence segment, comprising a capture sequence at the 3′ end of the inner primer sequence segment and a reverse complimentary sequence downstream from the capture sequence;
   wherein the 5′ end of the bumper sequence segment is connected to the 5′ end of the inner primer sequence segment.

In one embodiment, the primer comprises a cleavage site located between the bumper sequence segment and the capture sequence segment. In one embodiment, the cleavage site comprises one or more ribonucleotides that are cleavable by a RNase H enzyme. In one embodiment, the cleavage site comprises a single ribonucleotide. In one embodiment, the capture sequence segment has a higher melting temperature (Tm) than the bumper sequence segment. In one embodiment, the Tm of the capture sequence segment is about 2° C. to 7° C. higher, preferably 5° C. to 7° C. higher, than the Tm of the bumper sequence segment. In one embodiment, the bumper sequence segment anneals to the target polynucleotide region upstream of where the capture sequence segment anneals to the target polynucleotide region.

In another aspect, there is provided a kit for amplifying a target polynucleotide region of a nucleic acid molecule comprising, in one or more containers, at least two target-specific co-operative primers as described above; a thermostable polymerase; and a buffer.

In one embodiment, the at least two target-specific co-operative primers comprises: (a) a first primer that anneals to a first region of the target polynucleotide region; and (b) a second primer that anneals to a region of an extension product of the first primer.

In one embodiment, the nucleic acid molecule is a double stranded DNA, and wherein the second primer anneals to a second region of the target polynucleotide region on a strand complementary to the first region. In one embodiment, where the nucleic acid molecule is a double stranded DNA, the at least two target-specific co-operative primers comprises: (a) a first primer that anneals to a first region of the target polynucleotide region; (b) a second primer that anneals to a second region of the target polynucleotide region on the complementary strand; (c) a third primer that anneals to a third region of the target polynucleotide region; and (d) a fourth primer that anneals to a fourth region of the target polynucleotide region on the complementary strand.

In one embodiment, the kit further comprises two loop primers. In one embodiment, the buffer has a pH in the range of pH 6 to pH 9 and comprises a stabilization agent selected from the group consisting of BSA, glycerol, a detergent and mixtures thereof. In one embodiment, the buffer contains a monovalent salt having a concentration in the range of 0-500 mM. In one embodiment, the buffer comprises a divalent metal cation having a concentration of 0.5 mM-10 mM. In one embodiment, the buffer has a pH in the range of pH 6-pH 9, and comprises a monovalent salt having a concentration in the range of 0-500 mM, and a divalent metal cation having a concentration of 0.5 mM-10 mM and optionally a stabilizing agent selected from the group consisting of BSA, glycerol, a detergent and mixtures thereof. In one embodiment, the thermophilic polymerase has strand displacement activity and is active at temperatures greater than about 50° C. In one embodiment, the buffer further contains a single stranded binding protein (SSB) in the range of 0.5 ug to 2 ug per reaction. In one embodiment, the kit further comprises a ribonuclease (RNase) enzyme, preferably a is RNase H2 enzyme. In one embodiment, the kit further comprises deoxynucleotides (dNTPs).

In one embodiment, the kit comprises one or more of a fluorescent probe; a DNA binding dye; a PNA or BNA probe and a dye that recognizes PNA/BNA DNA complexes; or a methylene blue dye for cyclic voltammetry. In one embodiment, the kit comprises a RNase inhibitor.

In one embodiment, the kit comprises: (a) a first primer comprising SEQ ID No: 1; (b) a second primer comprising SEQ ID No: 2; (c) a first loop primer comprising SEQ ID No: 3; and (d) a second loop primer comprising SEQ ID No: 4.

In one embodiment, the kit comprises: (a) a first primer comprising SEQ ID No: 5; (b) a second primer comprising SEQ ID No: 6; (c) a first loop primer comprising SEQ ID No: 7; and (d) a second loop primer comprising SEQ ID No: 8.

In one embodiment, the kit comprises: (a) a first primer comprising SEQ ID No: 9; (b) a second primer comprising SEQ ID No: 10; (c) a first loop primer comprising SEQ ID No: 11; and (d) a second loop primer comprising SEQ ID No: 12.

In another aspect, there is provided a method of amplifying a target polynucleotide region of a nucleic acid molecule, comprising: contacting the nucleic acid molecule with: at least two target-specific co-operative primer as described above, and a thermostable polymerase; under a condition that promotes strand displacement amplification.

In one embodiment, the method further comprises cleaving the cleavage sites using a RNase H enzyme. In one embodiment, the method further comprises contacting the nucleic acid molecule with two loop primers. In one embodiment, the method further comprises contacting the nucleic acid molecule with a single stranded binding protein (SSB). In one embodiment, the method comprises: (a) combining the single stranded binding protein (SSB) with the thermostable polymerase, the at least two primers and the nucleic acid molecule in a reaction buffer at a first temperature; and (b) immediately or after a lag time at a temperature above 4° C. but below 70° C., performing an isothermal strand displacement amplification reaction at a second temperature, wherein the increase is determined with respect to the same mixture without the SBB.

In one embodiment, the method comprises performing PCR, qPCR, HDA, LAMP, RPA, TMA, NASBA, SPIA, SMART, Q-Beta replicase, or RCA. In one embodiment, the method further comprises isolating the amplified target polynucleotide region. In one embodiment, the method further comprises detecting the amplified target polynucleotide region using a fluorescent probe; a DNA binding dye; a PNA or BNA probe and a dye that recognizes PNA/BNA DNA complexes; or a methylene blue dye for cyclic voltammetry.

### BRIEF DESCRIPTION OF THE FIGURES

These and other features of the preferred embodiments of the invention will become more apparent in the following detailed description in which reference is made to the appended drawings wherein:

FIG. 1 shows a schematic of a cleavable co-operative primer (CCP) containing two oligonucleotide sequence segments with two different melting temperatures (Tm) and a single ribonucleotide located between the capture (F2) and the bumper (F3) sequences. The CCP also has a region (F1C) that is complementary to a target region of a nucleic acid molecule.

FIG. 2 shows a schematic diagram showing the annealing of the F2 capture oligonucleotide sequence of the Forward CCP (F-CCP) to its complimentary sequence in the target (F2C). The arrow indicates where F3 will anneal to. The higher Tm of the F2 region of the cooperative primer binds to its complementary sequence first, anchoring the primer to the target. This facilitates the bumper primer (F3) with a lower Tm to more readily bind to its complementary sequence even though the reaction temperature is significantly above the Tm of the F3 sequence.

FIG. 3 shows a schematic diagram showing the annealing of the F3 bumper sequence to its complimentary F3C sequence located upstream of the F2 capture primer. Arrow indicates direction of polymerization.

FIG. 4 shows a schematic diagram showing (A) the extension of the F2 capture sequence in a 5′-3′ direction and (B) the displacement of the F2 capture sequence strand by the F3 bumper primer extension (FIG. 4B). Arrow indicates direction of polymerization.

FIG. 5 shows a schematic diagram showing the displaced F2 strand and the binding of the capture and bumper sequences of the reverse cleavable co-operative primer (R-CCP) to the displaced F2 extended strand. Arrow indicates direction of polymerization.

FIG. 6 shows a schematic diagram showing the extension of the B2 capture sequence along the displaced F2 sequence strand. Arrow indicates direction of polymerization.

FIG. 7 shows a schematic diagram showing the continued extension of the B2 capture primer sequence strand past the RNase H cleavage site between F3 and F1C sequences. The B3 bumper primer next extends and displaces the extended B2 capture primer sequence strand. Arrow indicates direction of polymerization.

FIG. 8 shows a schematic diagram showing the ribonucleotide cleavage site (white arrow) formed by the extended B2 capture strand in FIG. 6 and the F2 capture strand in FIG. 4. The dsDNA is cleaved by RNase H2 on the strand containing the ribonucleotide following formation of dsDNA. Black arrow indicates direction of polymerization.

FIG. 9 shows a schematic diagram showing the displacement of the B2 capture strand by the B3 extension product. White arrow indicates dsDNA cleavage site.

FIG. 10 shows a schematic diagram showing the extension and release of the F2 extended strand (shown by arrow) after RNase H cleavage of the forward cooperative primer cleavage site. This product can now extend around the reverse co-operative primer cleavage site forming a loop and participate further in amplification. The B2 extension product generates a loop product which activates the cleavage site within the R-CCP primer between B3 and B1C.

FIG. 11 shows a schematic diagram showing the release of the F2 extension product (bottom) after RNase H cleavage and extension of the F2C strand shown by arrow in FIG. 10 which then forms a loop structure containing the cleavage site on the R-CCP strand.

FIG. 12 shows a schematic diagram showing the annealing of the F1C and F1 sequences forming a loop structure (top panel) which is extended in a 5′-3′ direction and the R-CCP primer binding to the liberated F2 extension product (bottom panel). A reverse Cooperative Primer binds to F2 Extension Product to generate double stranded product. Arrows indicate directions of polymerization.

FIG. 13 shows a schematic diagram showing the extension of the F1C/F1 loop around the R-CCP sequence on Product 1 (top panel) forming a cleavage site and the extension of the B2 capture primer sequence on Product 2 (bottom panel). White arrow indicates formation of dsDNA RNase H2 cleavage site at ribose site following extension of F1 strand. Reverse Cooperative Primer binding to F2 Extension Product generates double stranded product. Black arrow indicates direction of polymerization.

FIG. 14 shows a schematic diagram showing reverse Cooperative Primer binding to F2 Extension Product to generate double stranded product and start exponential amplification. The displacement of the lower strand of the F2 Extension Product by the B3 primer sequence extension (arrow) shown in the top panel of FIG. 13. The Product 2 resulting from RNase H cleavage at the dsDNA sites formed by the F-CCP and R-CCP primers. The B3 primer is extended and displaces the B2 strand of Product 2 (bottom).

FIG. 15 shows a schematic diagram showing F1C hybridizing to F1 of Product 2 and forming a loop structure (top panel). The bottom F1 strand is then extended forming a loop structure around the R-CCP primer cleavage site (second panel). Following cleavage at the R-CCP cleavage site the F2C strand is extended and displaces the BIC strand (third panel). The displacement allows BIC to form a loop with B1 which is subsequently displaced by BIC (bottom panel). White arrows indicate ribose base forming RNase H2 cleavage site on dsDNA. Black arrows indicate direction of polymerization.

FIG. 16 show a schematic diagram showing the F-CCP containing a cleavage site binding to F2C of the loop structure formed in FIG. 15 and is extended in a 5′-3′ direction towards the B1C/B1 loop structure (top panel). At the same time the B1C sequence is extended and displaces the B1C/B1 looped strand. The result is the formation of a long linear strand (bottom strand of bottom panel) which is subsequently cleaved (shown in FIG. 17). Arrows indicate direction of polymerization.

FIG. 17 shows a schematic diagram showing the F-CCP sequence being extended, cleaved and displaced by the B2 extension product forming Product 3 (top strand). Product 3 then enters into exponential amplification by formation of F1C/F1 and B1C/B1 loops with subsequent F-CCP and R-CCP annealing and extension. Backbone nicked by RNase H2 on the same strand of the ribonucleotide when double stranded DNA is formed.

FIG. 18A (Flu A 10⁴ Copies) and 18B (Beta Actin 10⁴ Copies) shows time to positivity for CCPSDA amplification for 10⁴ copies of influenza A/H1 and human Beta-actin.

FIG. 19 shows time to positivity for LAMP and CCPSDA amplification for 100 copies. CCPSDA amplification could detect 100 copies of Beta-actin for 8/8 replicates while traditional LAMP detected only 1/8. Amplification was measured using a BioRad CXF96 instrument and Eva green dye (Biotium, Inc.) detection of amplified DNA.

FIG. 20 shows time to positivity of traditional LAMP and CCPSDA amplification for 50 copies. CCPSDA amplification could detect 50 copies of Beta-actin for 8/8 replicates (squares) while LAMP failed to detect 50 copies (circles) in 8 replicates. Amplification was measured using a BioRad CXF96 instrument and Eva green dye (Biotium, Inc.) detection of amplified DNA.

FIG. 21 shows time to positivity of LAMP and CCPSDA amplification for 25 copies. CCPSDA amplification could detect 25 copies of Beta-actin for 5/8 replicates (squares) while traditional LAMP failed to detect 25 copies. Modified heated LAMP could detect 10 copies of Beta-actin for 2/8 replicates (data not shown). Amplification was measured using a BioRad CXF96 instrument and Eva green dye (Biotium, Inc.) detection of amplified DNA.

FIG. 22 shows time to positivity of Heated LAMP and CCPSDA amplification for 10 copies. CCPSDA amplification could detect 10 copies of Beta-actin for 3/8 replicates (squares) while modified heated LAMP detect 10 copies in 2/8 replicates (circles) and traditional LAMP failed to detect 10 copies. Amplification was measured using a BioRad CXF96 instrument and Eva green dye (Biotium, Inc.) detection of amplified DNA.

FIG. 23 shows specific and non-specification amplification of Heated LAMP and CCPSDA amplification. CCPSDA generates less non-specific products that appear later in the reaction compared with traditional LAMP and these products only appear after 50 minutes of amplification. SP, specific products; NSP, non-specific products; Green squares, CCPSDA specific amplification products; Red circles, LAMP specific products; Blue circles, no template LAMP; Orange squares, no template CCPSDA.

FIG. 24 shows the results for CCPSDA using two CCP primers and CCPSDA using four primers (two CCP primers and two loop primers). CCPSDA amplification with two CCP primers (right three plots) and with four primers (left three plots).

### DETAILED DESCRIPTION OF THE INVENTION

NAATs, especially real time PCR, multiplex PCR, and more recently isothermal amplification methods, have replaced conventional methods for detecting bacteria and viruses largely because these molecular tests detect 30 to 50% more positives. The movement towards isothermal amplification tests allows for the development of POC diagnostic tests, which should improve the detection and diagnosis of infections in clinical settings such as emergency rooms and walk in clinics, as well as non-clinical settings such as the home or in the field.

### Isothermal Amplification

Various amplification techniques have been developed that require multiple steps and more than a single temperature. Transcription-Mediated Amplification (TMA) employs a reverse transcriptase with RNase activity, an RNA polymerase, and primers with a promoter sequence at the 5′ end. The reverse transcriptase synthesizes cDNA from the primer, degrades the RNA target, and synthesizes the second strand after the reverse primer binds. RNA polymerase then binds to the promoter region of the dsDNA and transcribes new RNA transcripts, which can serve as templates for further reverse transcription. The reaction is rapid and can produce 10E9 copies in 20-30 minutes. This system is not as robust as other DNA amplification techniques. This amplification technique is very similar to Self-Sustained Sequence Replication (3SR) and Nucleic Acid Sequence Based Amplification (NASBA), but varies in the enzymes employed. Single Primer Isothermal Amplification (SPIA) also involves multiple polymerases and RNaseH. First, a reverse transcriptase extends a chimeric primer along an RNA target. RNaseH degrades the RNA target and allows a DNA polymerase to synthesize the second strand of cDNA. RNaseH then degrades a portion of the chimeric primer to release a portion of the cDNA and open a binding site for the next chimeric primer to bind and the amplification process proceeds through the cycle again. The linear amplification system can amplify very low levels of RNA target in roughly 3.5 hrs. The Q-Beta replicase method is a probe amplification method. A probe region complementary or substantially complementary to the target of choice is inserted into MDV-1 RNA, a naturally occurring template for Q-Beta replicase. Q-Beta replicates the MDV-1 plasm id so that the synthesized product is itself a template for Q-Beta replicase, resulting in exponential amplification as long as there is excess replicase to template. Since the Q-Beta replication process is so sensitive and can amplify whether the target is present or not, multiple wash steps are required to purge the sample of non-specifically bound replication plasmids. The exponential amplification takes approximately 30 minutes; however, the total time including all wash steps is approximately 4 hours.

Several isothermal amplification techniques have been developed to circumvent the need for temperature cycling. Strand displacement amplification (SDA) was developed by Walker et al. in 1992. This amplification method uses two sets of primers, a strand displacing polymerase, and a restriction endonuclease. The bumper primers serve to displace the initially extended primers to create a single-strand for the next primer to bind. A restriction site is present in the 5′ region of the primer. Thiol-modified nucleotides are incorporated into the synthesized products to inhibit cleavage of the synthesized strand. This modification creates a nick site on the primer side of the strand, which the polymerase can extend. This approach requires an initial heat denaturation step for double-stranded targets. The reaction is then run at a temperature below the melting temperature of the double-stranded target region. Products 60 to 100 bases in length are usually amplified in 30-45 minutes using this method.

SDA was the first isothermal amplification method described and involves restriction endonuclease nicking of a recognition site in an unmodified strand, followed by strand-displacing polymerase extension of the nick at the 3′ end, which displaces the downstream strand. The displaced strand can then act as a target for an antisense reaction, ultimately leading to exponential amplification of DNA. Since its development, it has been improved using approach such as hyperbranching and applied for whole genome analysis of genetic alterations.

Rolling circle replication was first characterized as the mechanism through which viral circular genomes are replicated. Subsequently, it has been applied as both an exponential DNA amplification tool (100-fold increase in DNA) and a rapid signal amplification tool (100-fold signal amplification). In this approach, a small circular piece of DNA is primed by the target, after which a strand displacement polymerase enzyme continues around the circular DNA, displacing the complementary strand. Ultimately, the synthesized DNA remains attached to the circle as more DNA is generated, generating 10⁹ or more copies of the circle within 90 minutes. RCA has been applied for the detection of point mutations in human genomic DNA.

Recombinase polymerase amplification (RPA) is one of the more recent isothermal DNA amplification techniques, involving a mixture of three enzymes; namely, a recombinase, a single stranded DNA-binding protein (SSB), and a strand displacing polymerase. The recombinase enzyme is able to scan and target primers to their complementary sequence in the double-stranded target DNA, at which time the SSB binds and stabilizes the primer-target hybrid, allowing the strand-displacement polymerase to initiate DNA synthesis. Using this approach, DNA amplification can be achieved within 10 to 20 minutes, showing a high sensitivity and specificity. RNA amplification is also possible, as shown through the reverse transcriptase RPA (RT-RPA) assay targeting coronavirus. In a recent report, Wang et al. demonstrated detection of Feline herpesvirus 1 (FHV-1) within 20 minutes, at a detection level of 100 copies. These reports support that RPA is a powerful tool for the rapid detection of DNA and RNA targets.

Helicase dependent amplification (HDA) is a method where DNA is replicated in vivo by DNA polymerase in combination with numerous accessory proteins, including DNA helicase to unwind the double-stranded DNA. In HDA, a helicase is included in the amplification mixture so that thermocycling is not required for amplification. The single-stranded DNA intermediate for primer binding is generated by the helicase enzyme, as opposed to PCR where a heat denaturing step is required. HDA has been applied in numerous biosensors for the detection of multiplex pathogen detection, and has promise for use in disposable POC diagnostic devices, such as for the detection of Clostridium difficile.

LAMP is currently one of the most widely used and robust isothermal amplification techniques for amplifying either DNA or RNA sequences which is based on a strong strand-displacement polymerase combined with four to six primers. These primers recognize several specific regions in the target DNA, while two of the primers form loop structures to facilitate subsequent rounds of amplification. In this way, you achieve highly efficient isothermal amplification. Since the LAMP reaction is so robust, an extremely large amount of DNA is generated; accordingly, pyrophosphate ions (a biproduct of the amplification) are generated, yielding a cloudy precipitate (magnesium pyrophosphate) that can be used to determine whether amplification has occurred. Using this approach, 1 to 10 copies of DNA can be amplified to 10⁹ to 10¹⁰ copies within 30 to 60 minutes, showing excellent sensitivity and specificity. LAMP however suffers from poor specificity due to primer dimer formation and amplification of non-specific products. In addition, multiplex LAMP assays (M-LAMP) can be established, as has been shown for influenza A/H1, A/H3, and Influenza B, as well as Respiratory Syncytial Virus (RSV) A and B, with rapid diagnosis and single genome copy sensitivity.

As opposed to the DNA amplification methods discussed above, SMART or simple method to amplify RNA targets is based on signal amplification after formation of a three-way junction (3WJ) structure; the actual DNA or RNA target is not amplified. Two oligonucleotide probes are included in the reaction, both of which have complementary sequences to the DNA or DNA target as well as a smaller region that is complementary to the other probe. The two probes are brought into proximity upon binding to their target, at which time the 3WJ is formed. Upon formation of the 3WJ, polymerase can extend the target-specific oligonucleotide, forming a double stranded T7 promoter region; this results in constant production of RNA in the presence of target DNA, which can be detected in a real-time manner. SMART has been applied clinically to detect marine cyanophage DNA in marine and freshwater environments.

Factors that adversely affect the outcome of amplification methods are numerous and include inhibitors of polymerase activity and other components found in clinical specimens that reduce amplification efficiency, reduce amplification efficiencies due to secondary structure of primers or template, and template-independent amplification resulting from primer-dimer formation that decreases amplification efficiency and specificity leading to false positives. The negative effects are amplified at room temperature following the setup of reaction mixtures before they are moved to the amplification temperature presenting specificity problems for labs batching a large number of specimens. This can occur when a large number of reactions are prepared for a single run resulting in holding of reactions at room temperature. This is a common occurrence in large laboratories that process high specimen volumes and where batch processing is required for high throughput of results. High throughput is therefore often negatively impacted by set up at room temperature and key requirements for molecular diagnostic testing including consistency, reproducibility and accuracy can be negatively impacted. RNase H2 primers that contain a single ribonucleotide near the 3′-terminus and containing a phosphothioate nucleotide blocked have been used.

To further accelerate DNA detection assays, signal amplification approaches are becoming more common. This involves an early specific-sequence detection step followed by an exponential cascade of DNA production that is no longer reliant on the initial target being present. Examples of signal amplification include Nucleic Acid Sequence Based Amplification (NASBA), Transcription Mediated Amplification (TMA) and SMART.

These and other amplification methods are discussed in, for example, Van Ness. J, et al. PNAS 2003 100 (8): 4504-4509; Tan, E., et al. Anal. Chem. 2005, 77:7984-7992; Lizard, P., et al. Nature Biotech 1998, 6:1197-1202, the entire content of which is incorporated herein by reference.

Primers containing a single ribonucleotide which is cleavable by RNase H and a blocked 3′-terminus have been used to decrease primer dimer formation and reduce non-specific amplification. RNase H binds to RNA/DNA duplexes and cleaves at the RNA base and the blocking group from the end of the primer. The requirement of the primer to first hybridize with the target sequence forming dsDNA before RNase H cleavage and activation eliminates the formation of primer dimers and reduces non-specific amplification. RNase H-dependent PCR or rhPCR using these blocked cleavable primers has been used for the detection of single nucleotide polymorphisms (SNPs).

### Co-Operative Primers

Isothermal amplification of a nucleic acid sequence requires specificity in the early stages of amplification combined with exponential DNA amplification for maximal sensitivity of DNA detection. However, despite the good sensitivity and specificity of Loop-mediated isothermal amplification (LAMP), it is adversely affected by primer-dimer formation which decreases both sensitivity and specificity. Primer dimer formation can lead to non-specific amplification products that decrease the limit of detection of both PCR and LAMP. A variety of hot starts have been used for PCR, cooperative primers have been used for PCR and RNase H-cleavable primers and SSB proteins have been used to reduce non-specific amplification products in both PCR and LAMP.

Co-operative primers containing two nucleotide sequences connected by a polyethylene glycol linker and complimentary to a target gene to be amplified can be used in PCR to prevent primer dimer formation and reduce the amount of non-specific amplification products. Cooperative primers containing a probe sequence can also be used to generate a higher fluorescent signal following amplification.

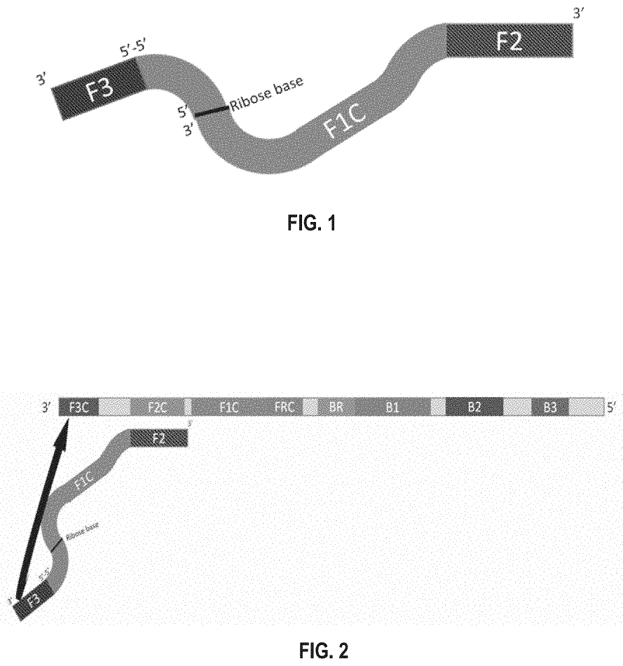

FIG. 1 is a schematic of an example target-specific co-operative primer (CCP) for amplifying a target polynucleotide region of a nucleic acid molecule. In this example, a forward CCP is shown. A reverse CCP has similar structure and sequence regions as a forward CCP.

In some embodiments, the co-operative primer comprises a 3′ to 5′ bumper sequence (F3, B3) attached to a 5′ to 3′ inner primer sequence. The 5′ end of the bumper sequence is connected to the 5′ end of the inner primer sequence, such that the primer contains 2 sequence segments that are in opposite direction to each other.

In some embodiments, the inner primer sequence has a target region that is complementary to a target sequence of a nucleic acid molecule. Examples of nucleic acid molecules to be amplified include single and double stranded DNA, as well as RNA. The 3′ end of the inner primer sequence comprises a capture sequence (F2, B2). In some embodiments, the inner primer sequence comprises a reverse complimentary sequence (F1C, B1C) downstream of the capture sequence. The bumper sequence is in a 3′-5′ direction, opposite to the capture sequence which is in a 5′-3′ direction. Therefore, a co-operative primer has two 3′ ends, one on the capture sequence (F2, B2) and one on the bumper sequence (F3, B3). Since the primer has two 3′ ends, polymerization occurs from both ends of the primer.

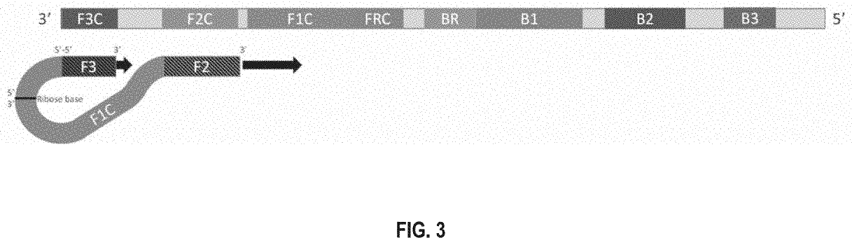

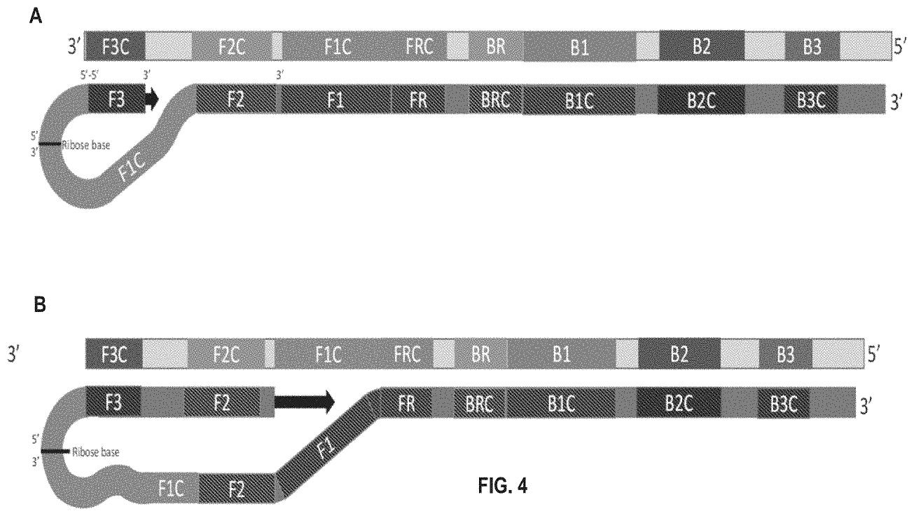

The capture sequence has a higher melting temperature (Tm) than the bumper sequence. Since the capture sequence has a higher Tm, it will anneal to a target sequence of a nucleic acid molecule first, before the bumper sequence anneals to its complementary target sequence (see FIG. 2). In some embodiments, the co-operative primer has a high Tm capture sequence and a low Tm bumper sequence. In one embodiment, the capture sequence has a Tm that is 1° C. to 10° C. higher than the Tm of the bumper sequence, preferably 2° C. to 7° C. higher, more preferably 5° C. to 7° C. higher. The bumper sequence anneals to the target nucleic acid molecule upstream of the capture sequence. Since the primer contains 2 sequence segments that are in opposite direction to each other, the primer loops back on itself in order for both the bumper and capture sequences to anneal to the target nucleic acid molecule (see FIG. 3). As polymerization occurs from both ends, polymerization from the 3′ end of the bumper sequences displaces the capture sequence as well as its extension product (see FIG. 4).

In some embodiments, a co-operative primer contains one cleavage site, comprising one or more ribonucleotides, located between the bumper and capture sequences. The cleavage site is cleavable by a ribonuclease enzyme, such as a RNase H enzyme. Examples of ribonuclease enzymes include, RNase H1 and RNase H2 enzymes. In one embodiment, the co-operative primer contains one cleavage site comprised of a single ribonucleotide, while the rest of the primer are deoxynucleotides.

In some embodiments, nucleic acid sequences are amplified by isothermal strand displacement amplification (iSDA) using a preparation comprising at least two co-operative primers (CCP), a thermostable strand displacement DNA polymerase polymerase, and a buffer. Since the isothermal strand displacement amplification is mediated by CCP primers, the amplification process is also called CCPSDA. The products of the amplification feed back into the iSDA to improve the lower limit of detection and shorten the time-to-positivity.

In one embodiment, CCPSDA uses one forward (F-CCP) and one reverse (R-CCP) cleavable cooperative primer. The F-CCP binds to a first target sequence of a target nucleic acid molecule, such as a strand of DNA. The R-CCP binds to a second target sequence on the extension product of the F-CCP. The R-CCP can also bind to a second target sequence on the complementary target nucleic acid molecule, such as the complementary strand of DNA.

In an alternative embodiment, four CCP primers are used (two F-CCP and two R-CCP). The two forward primers bind to one strand and the two reverse primers would bind to the complimentary strand generating additional products to enter into the exponential amplification phase.

In some embodiments, CCP primers are used together with two loop primers (LF and LB) and a thermostable strand displacement DNA polymerase for target amplification. In some embodiments where two loop primers are used to speed up the reaction, the loop primers increase the amount of target DNA that is exponentially amplified. Referring to FIG. 3, in one embodiment, the first loop primer is complimentary to the first displaced strand between the F2C and F1C regions, and the second loop primer is complimentary to the region between B2C and B1C. Using two loop primers in addition to the CCP primers speeds up the reaction, as opposed to just the CCP primers. Specific nucleic acid sequences of viral, bacterial, fungal pathogens, or eukaryotic DNA (see Table 1) can be amplified and generate a specific product for detection using a variety of DNA binding dyes or DNA-specific probes.

TABLE 1
Examples of Oligonucleotides used
for Co-operative Primers

| | |
|---|---|
| Target | Primer sequence |

direction: 5′-3′ unless otherwise specified
ribonucleotide base is indicated as (r n)

| | |
|---|---|
| Human | F-CCP: |
| B-actin | 3′-ACCCCATGAAGTCCCACT-5′ |
| | 5′-GCTCCTCGG(rG)AGCCACA |
| | CGCAGCTCATTGTAGAGCACGGC |
| | ATCGTCACCAAC-3′ |
| | (SEQ ID NO: 1) |
| | R-CCP: |
| | 3′-GCAACGATAGGTCCGACA-5′ |
| | 5′-AGGCCCCC(rC)TGAACCCC |
| | AAGGCCAACCCATGGCTGGGGTG |
| | TTGAAGGTCT-3′ |
| | (SEQ ID NO: 2) |
| | LF: |
| | AGATTTTCTCCATGTCGTCCCA |
| | (SEQ ID NO: 3) |
| | LB: |
| | CGAGAAGATGACCCAGATCATGT |
| | (SEQ ID NO: 4) |
| Influenza | F-CCP: |
| A/H1 | 3′-TCCCGTAAAACCTATTTCGCA-5′ |
| | 5′-TGACACCT(rC)CTTGGCCCCAT |
| | GGAACGTTGAAATGGGGACCCGAACA |
| | ACATGG-3′ |
| | (SEQ ID NO: 5) |
| | R-CCP: |
| | 3′-AGCCAGATCAAACACGGTGA-5′ |
| | 5′-CTAAGCT(rA)TTCAACTGGTGC |
| | ACTTGCAAGGCTTCTGTGGTCACTGT |
| | TCCCATCC-3′ |
| | (SEQ ID NO: 6) |
| | LF: |
| | 5′-TGAGCTTCTTGTATAGTTTAACT |
| | GC-3′ |
| | (SEQ ID NO: 7) |
| | LB |
| | 5′-TGCATGGGCCTCATATACAACA-3′ |
| | (SEQ ID NO: 8) |
| Influenza | F-CCP: |
| A/H3 | 3′-TACTCCGGGTACGTTGAC-5′- |
| | 5′-CTGTGCT(rG)GGAATCAGCAAT |
| | CTGCTCACACAATAGGATGGGGGCTG |
| | TAACCAC-3′ |
| | (SEQ ID NO: 9) |
| | R-CCP: |
| | 3′-ATACCTCGTTTACCGACCTAG-5′ |
| | 5′-GTCTCAT(rA)GGCAGATGGTGG |
| | CAACACTTAGCTGTAGTGCTGGCCAA |
| | AACC-3′ |
| | (SEQ ID NO: 10) |
| | LF: |
| | AATCTGCTCACATGTTGCACA |
| | (SEQ ID NO: 11) |
| | LB: |
| | CATTAATAAAACATGAGAACAGAAT |
| | (SEQ ID NO: 12) |

F-CCP is forward co-opcrative primer
R-CCP is reverse co-operative primer
LF is forward loop primer
LB is backward loop primer

The F-CCP and R-CCP bind to regions of a target genomic DNA consisting of 45-75 nt in length. The two CCP primers contain a 3′-capture oligonucleotide sequence (F2 or B2) and an upstream bumper oligonucleotide sequence (F3 or B3) separated by a ribonucleotide (see FIG. 1). The capture oligonucleotide sequence has a melting temperature (Tm) that is 5-7 degrees above the Tm of the bumper oligonucleotide sequence. The capture sequence (F2, B2) of the CCP primer binds first before the bumper oligonucleotide sequence (F3, B3) binds.

After the F2 capture sequence and the F3 bumper sequence anneals to complementary sequences of the target genomic DNA, they are both extended in a 5′-3′ direction by a thermostable polymerase (FIG. 3). The F3 3′ end is extended in a 5′-3′ direction and displaces the extension product from the F2 3′ end which is also extended in a 5′-3′ direction (see arrows in FIGS. 3 and 4).

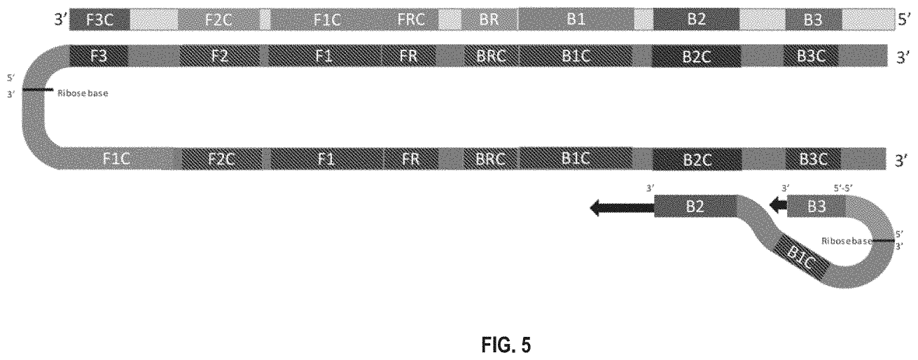

The R-CCP primer then binds to the 3′ end of the displaced strand in two stages (FIG. 5): 1) the B2 capture sequence binds first, and then 2) the B3 bumper sequence, which has a lower Tm than B2, binds second.

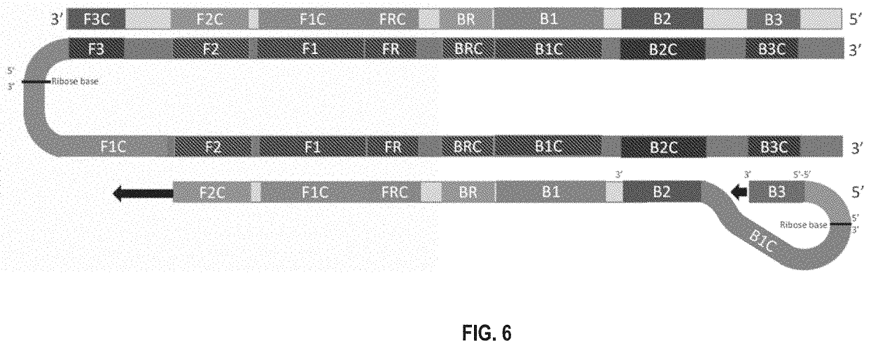

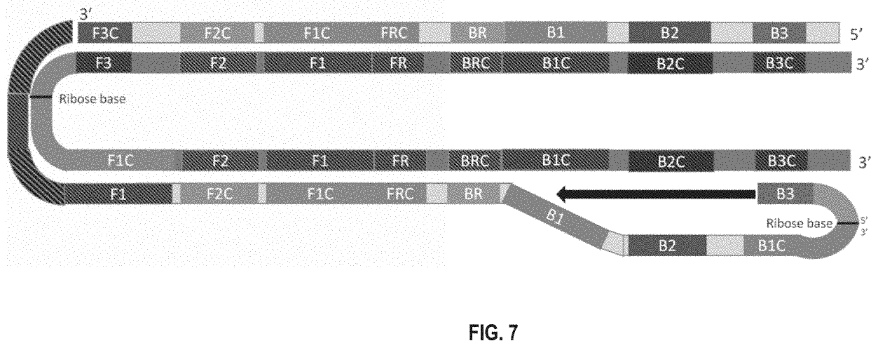

The 3′ end of B2 is extended in a 5′-3′ direction (FIG. 6) and the 3′ end of B3 is then extended in a 5′-3′ direction, displacing the extension product from B2 3′ end (FIG. 7).

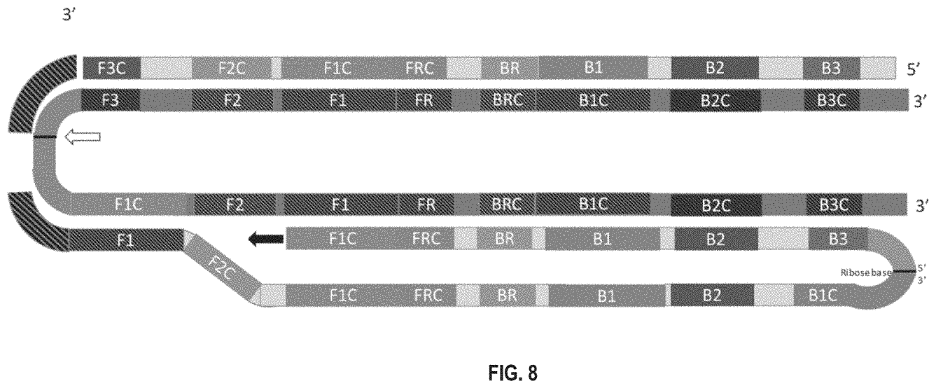

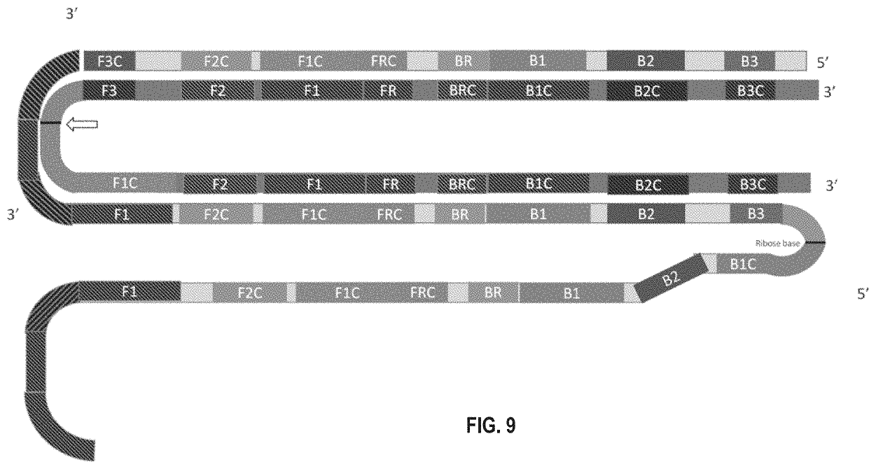

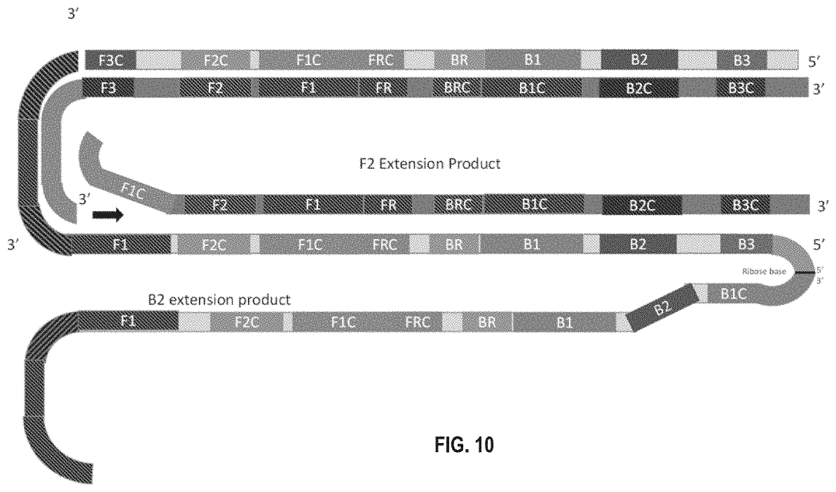

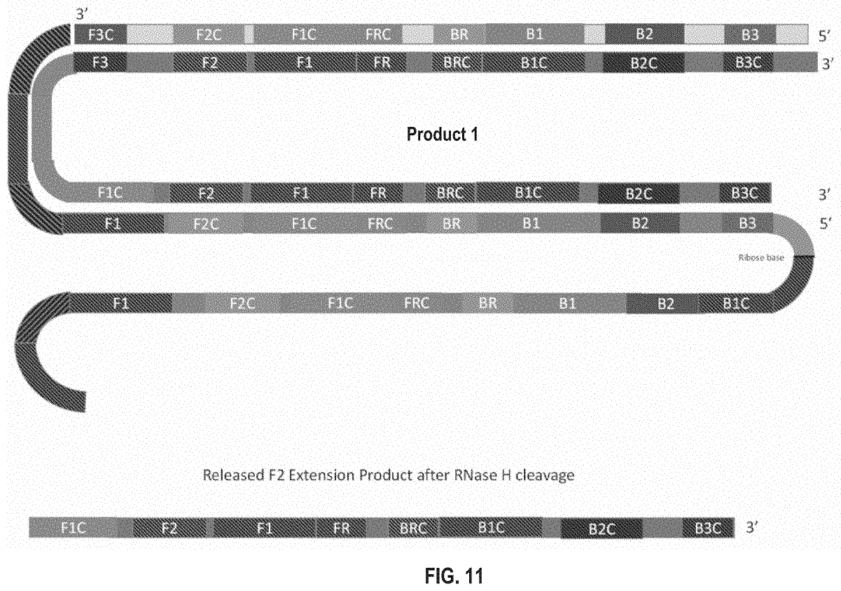

The B2 extension product is extended in a 5′-3′ direction along the length of the F2 extension product and past the ribose base on the F-CCP primer sequence (FIG. 8). The B2 extension product stops polymerizing when it reaches the 5′-5′ linkage on the F3 sequence. The full length of the B2 extension product is displaced by the B3 extension product as it also extends until the 5′-5′ linkage of the F3 sequence (see FIG. 9). With the B2 extension product extending past the ribose base, the ribose base acts as an RNase H cleavage site and the dsDNA is cleaved by RNase H (see FIG. 9), exposing a new 3′ end for further extension in a 5′-3′ direction, which displaces the F2 extension product (see FIG. 10) and thereby releasing the F2 extension product as shown as the bottom strand in FIG. 11.

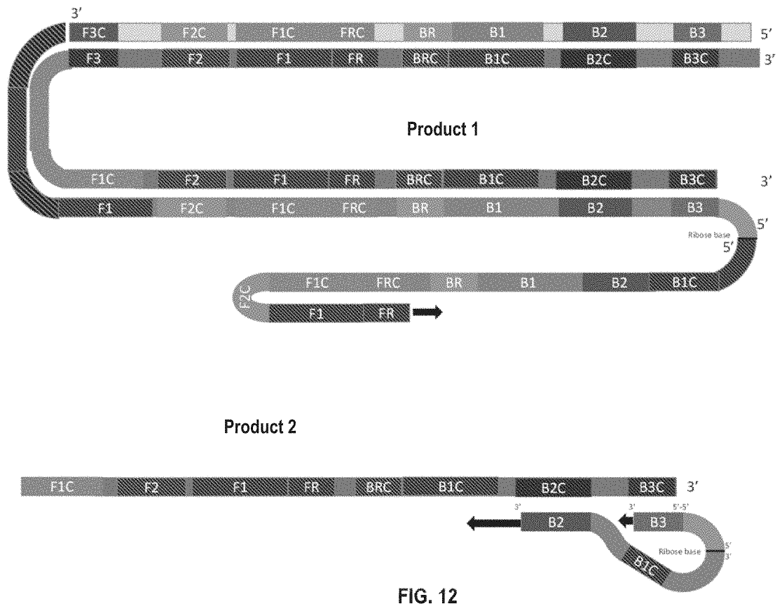

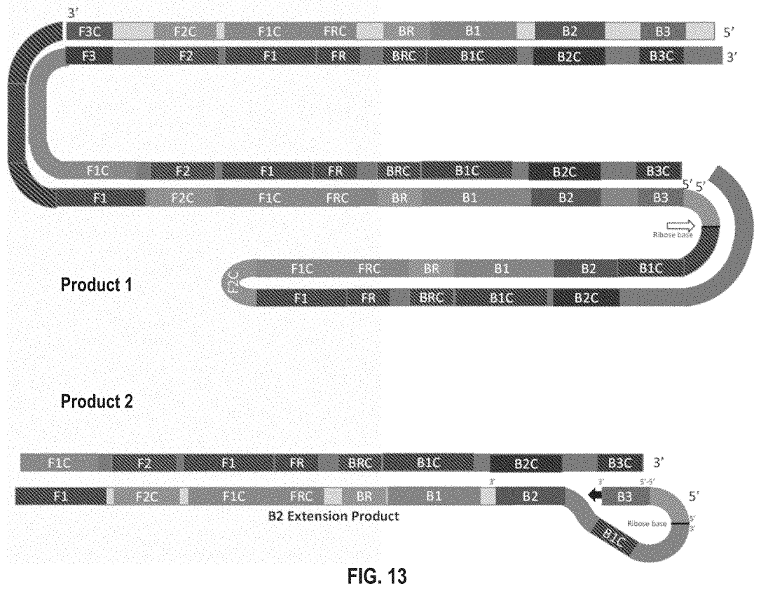

Turning to FIG. 12, the B2 extension product forms a loop is at the F2C sequence of the B2 extension product, by the hybridization of F1 sequence of the B2 extension product with F1C sequence of the B2 extension product (Product 1, top panel of FIG. 12). This allows the 3′ end of F1 to be extended in a 5′-3′ direction back along the length of the B2 extension product around past the ribose base of R-CCP (FIG. 13). RNase H then cleaves R-CCP of Product 1, exposing a 3′ end for extension, thereby displacing and releasing the looped B2 extension product (shown in FIG. 13) for exponential amplification (not shown).

A second R-CCP binds to the released F2 extension product (Product 2) and is then extended in a 5′-3′ direction (bottom panel of FIG. 12), forming a complimentary strand to the released F2 extension product (bottom panel of FIG. 13).

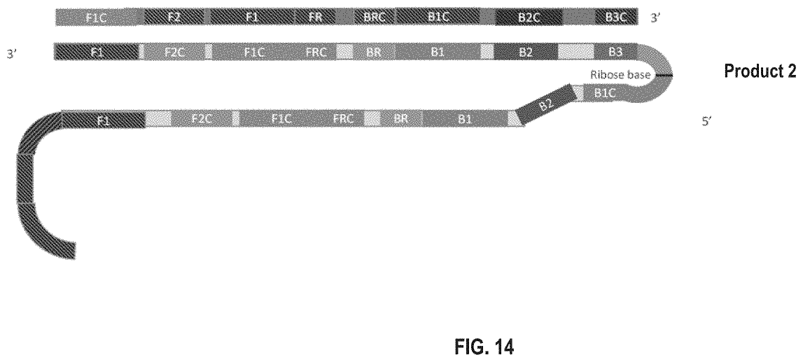

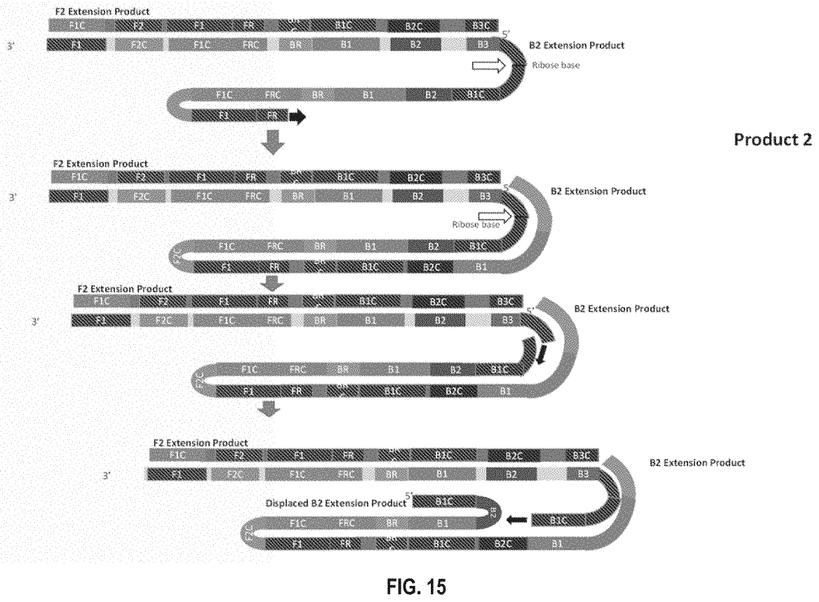

Same as before, the B2 extension product from the second R-CCP (Product 2) is then displaced by the extension of B3 in a 5′-3′ direction (see arrow in bottom panel of FIG. 13) and this displaced B2 extension product (Product 2, FIG. 14) forms a loop at F2C by the hybridization of F1 sequence with F1C sequence (FIG. 15, top panel). This loop is extended in a 5′-3′ direction past the ribonucleotide cleavage site of the second R-CCP, resulting in cleavage by RNase H (FIG. 15). The cleaved strand is then extended in a 5′-3′ direction from B3 (see arrow in third panel of FIG. 15) displacing the B2 extension product which forms a loop at B2 by the hybridization of the B1 sequence to the B1C sequence (bottom panel of FIG. 15).

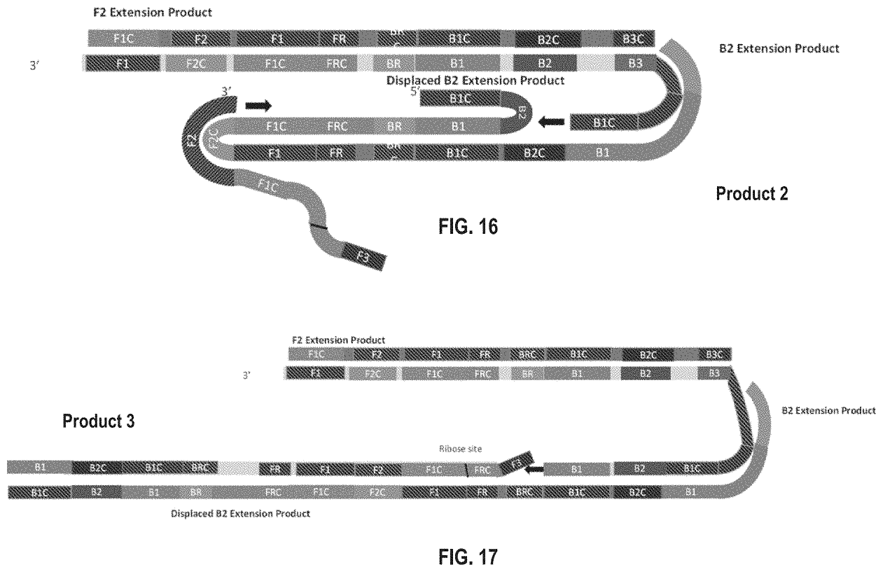

A second F-CCP primer then binds to the F2C loop and extends towards BIC and the B2 loop (FIG. 16). This extension product stops at the 5′-terminus of B1C and is displaced forming a long linear dsDNA (FIG. 17). This dsDNA is cleaved by RNase H at the ribonucleotide cleavage site and displaced by extension of the B1 terminal strand. The displaced strand then forms a loop at F2 by F1C hybridizing with F1 which acts as a template to initiate a further round of amplification. Both displaced strands are then amplified with the F-CCP and R-CCP primers and the cycle is repeated.

### Kits and Reagents

In some embodiments, a kit for amplifying a target polynucleotide region of a nucleic acid molecule includes at least two cleavable co-operative primers (at least one forward and one reverse co-operative primer), a thermostable polymerase, and a buffer in one or more containers. The thermostable polymerase has strand displacement activity and is active at temperatures in the range of 50-80° C. In one embodiment, the kit contains two cleavable co-operative primers, while in other embodiments the kit contains two forward co-operative primers and two reverse co-operative primers. In some embodiments, the kit further comprises dNTPs, RNase H enzymes, loop primers, single stranded binding proteins (SSBs), or combinations thereof.

In one embodiment, single stranded binding proteins (SSBs) are added to decrease background generated by primer dimer amplification. The SSBs can be provided in the buffer at a range of 0.5 ug to 2 ug per reaction.

To detect the amplified nucleic acid molecules, various DNA detection methods can be used. For example, the amplification products can be detected by fluorescent signal detection using a fluorescent probe. The amplification products can be visually detected using a DNA binding dye, by specific visual detection of DNA using a PNA or BNA probe and a dye that recognizes PNA/BNA DNA complexes. Other examples of detecting amplification products include using methylene blue dye with cyclic voltammetry.

In some embodiments, the kit is for amplifying target DNA and/or RNA. In some embodiments, the kit has a RNase inhibitor. In one embodiment, the RNase inhibitor is from NEB™ (RNase inhibitor, Murine cat #M0314L). In one embodiment, the RNase inhibitor is from Promega™ (RNasin Native (cat #N2215) and RNasin Recombinant (cat #N2515). In the case of amplification of RNA from different pathogens, it is prudent to have an inhibitor of RNAses in the reaction mixture. RNAses are exceedingly ubiquitous and can be found contaminating surfaces and/or plastics which are used in manufacturing, or can be found in crudely purified specimens. The use of an RNAse inhibitor prevents the degradation of the RNA targets (RNA genome or even RNA transcripts) during the amplification. Amplification of RNA targets is impacted if the RNAse inhibitor is not present and the reaction is contaminated with RNAses. In some embodiments, the presence of an RNAse inhibitor improves the ability to detect RNA targets in situations where a total nucleic acid extraction (and thus removal of RNAses) cannot be performed prior to amplification and detection of a target. In some embodiments of the methods of amplifying a target polynucleotide region of a nucleic acid molecule described herein, the method comprises pretreatment with an RNAse inhibitor prior to introducing the primers described herein to the reaction mixture for amplification.

In one embodiment, the kit is a point of care diagnostic device. Examples of point of care diagnostic device are found in WO2016/0004536 (PCT/CA2015/050648) and WO2017/117666 (PCT/CA2017/000001), the entire contents of which are incorporated herein by reference.

### EXAMPLES

### Example 1—Detection of DNA Using CCPSDA

This example outlines a method of demonstrating the use of CCP primers in an isothermal strand displacement (SDA) amplification reaction.

To evaluate the functionality of CCP primers in SDA we amplified two different gene targets including the beta-actin gene from human genomic DNA and influenza A/H1gene. The primers (F-CCP, R-CCP, LF, LB) used for the assays to be used in the evaluations are detailed in Table 1. The CCPSDA reactions utilizing each set of primers were held at 25° C. (room temperature) for 0 to 2 hours prior to testing to allow primer dimer formation. After the room temperature hold, all reactions were run at 63° C. for 30 minutes in a BioRad CFX96. Signal amplification in all of these reactions will be performed with 1× Eva green added to the reaction (see: Biotechnology Letters, December 2007, volume 29, Issue 12, pp 1939-1946, the content of which is incorporated herein by reference.)

For CCPSDA amplification using CCP primers, the primer mix and template were heated to 94° C. for 4 mins, kept at 66° C. for a few minutes and cooled to room temperature just prior to addition to the reaction mixture. The primer/template mix was added to the reaction mix containing dNTP, Eva green, Bst 3.0, RNase H2 and amplified for 30 minutes at 63° C. on the BioRad CFX96.

For R-CCP and F-CCP primers the titration included 0 μM, 0.2 μM, 0.4 μM, 0.8 μM and 1.2 μM/reaction. For the second set of primers, LF and LB, the titration included 0 μM, 0.2 μM, 0.4 μM, 0.8 μM and 1.2 μM/reaction.

The 25 μL Eva green reaction mixtures included: 12.5 μL of 2× Master Mix (1× is 20 mM Tris-HCl, 10 mM (NH₄)₂SO₄, 150 mM KCl, 2 mM MgSO₄, 0.1% Tween 20 pH=8.8 for LAMP and Isothermal Amplification Buffer II (NEB) for iSDA), 0.6 mM dNTPs, 0.8 μM F-CCP and R-CCP primers, 0.4 μLF and LB primers, 6U Bst 3.0 enzyme, 0.6 mM RNase H2 (IDT) (for RNase H2 control, buffer D will be used), 2 μL sample (either 20 ng/mL human gDNA or 2.5 ng/mL human gDNA or Influenza A RNA, and Nuclease free water to 25 μL.

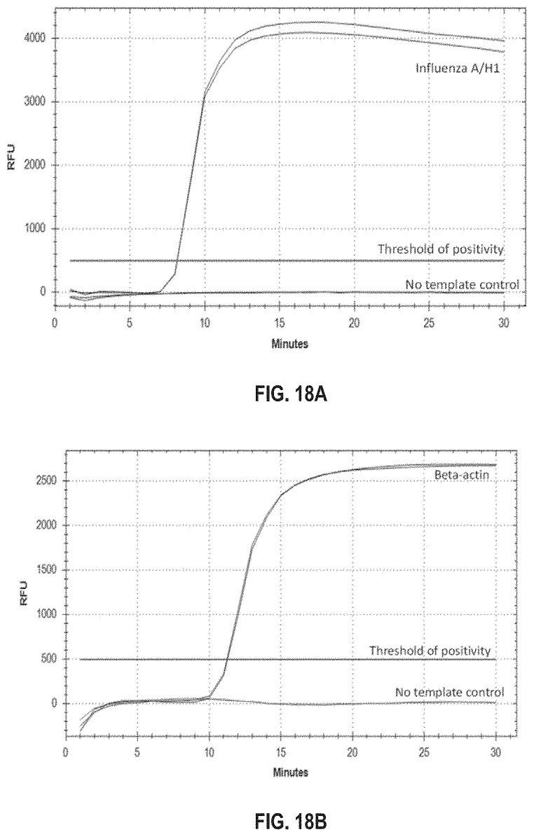

The results are shown in FIG. 18 for 10⁴ target copies of influenza A/H1 (FIG. 18A) and 10⁴ target copies of human beta-actin (FIG. 18B).

### Example 2—CCPSDA Shows a Reduced Time to Reach Threshold Amplification Levels Compared to LAMP

The following example demonstrates the improved sensitivity of CCPSDA compared with traditional LAMP.

The following example demonstrates the reduced time of CCPSDA amplification to reach threshold amplification levels compared with LAMP using six unmodified primers. The increase in the rate of amplification is measured by time taken to reach threshold amplification.

CCPSDA and LAMP reactions were performed at 63° C. The human Beta-actin CCP primers are listed in Table 1 and the LAMP primers are listed in Table 2.

TABLE 2
Human Beta-Actin LAMP Primers

| SEQ ID | | |
|---|---|---|
| No. | Name | Sequence (5′-3′) |
| 13 | FIP | GAGCCACACGCAGCTCATTGT |
| | | ACACGGCATCGTCACCAAC |
| 14 | BIP | CTGAACCCCAAGGCCAACCGG |
| | | CTGGGGTGTTGAAGGTC |
| 15 | F3 | CCCTGAAGTACCCCATCGA |
| 16 | B3 | ACAGCCTGGATAGCAACGT |
| 3 | LF | AGATTTTCTCCATGTCGTCCCA |
| 4 | LB | CGAGAAGATGACCCAGATCATGT |

For CCPSDA amplification using CCP primers, the primer mix and template were heated to 94° C. for 4 mins, kept at 66° C. for a few minutes and cooled to room temperature just prior to addition to the reaction mixture. The primer/template mix was added to the reaction mix containing dNTP, Eva green, Bst 3.0, RNase H2 and amplified for 30 minutes at 63° C. on the BioRad CFX96.

LAMP reactions were performed at 63° C. in replicates of 8 using 1×AMP Buffer II includes: 20 mM Tris-HCl, 10 mM (NH₄)₂SO₄, 150 mM KCl, 2 mM MgSO₄, 0.1% Tween® 20, pH 8.8 @ 25° C. Reactions were performed in 25 μL volumes and consisted of 8 U Bst 3.0 DNA polymerase (New England Biolabs, Ipswich, Mass.), 20 ng/5 μL, human genomic DNA (Roche Cat. No. 11 691 112 001) and F3 and B3 primers 0.2 μM, LF and LB primers 0.4 μM F1P and B1P primers 1.6 μM, as shown in Table 2, dNPTs 1.4 mM, and Eva Green dye.

The primers (Integrated DNA Technologies, Coralville, Iowa) were added to an amplification reaction (20 mM Tris pH 8.8 at 25° C., 10 mM (NH₄)₂SO₄, 2 mM MgSO₄) and supplemented with additional 6 mM MgSO₄, 0.01% Tween-20 and 1.4 mM dNTPs.

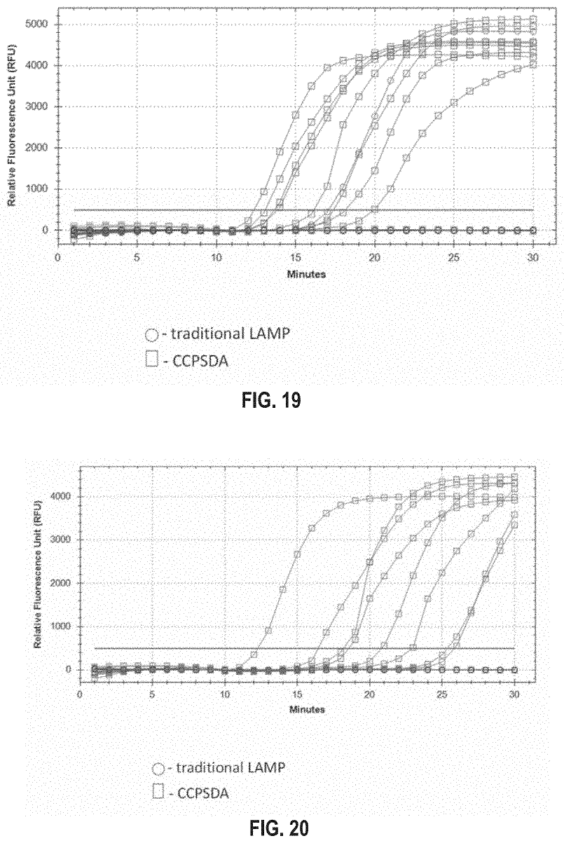

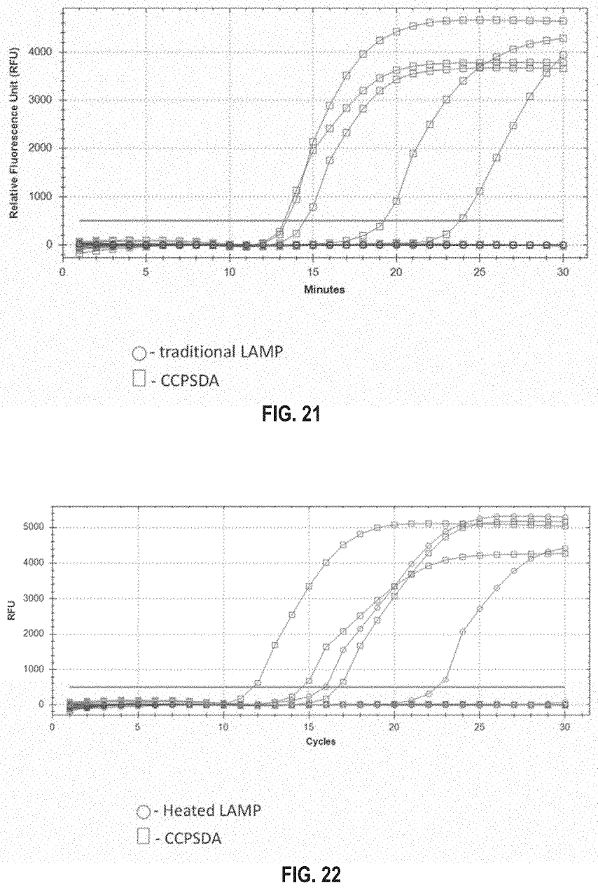

The results are shown in FIG. 19 for 100 target copies, FIG. 20 for 50 target copies, FIG. 21 for 25 target copies, and FIG. 22 for 10 target copies.

The time to positivity and the numbers positive/number tested for the various target copy numbers are summarized in Tables 3 and 4. For 10 target copies the time to positivity for CCPSDA was 12.4, 15.6, and 17 minutes compared to 16 and 23 minutes for Heated LAMP (FIG. 22). Traditional LAMP without the heating step failed to show amplification signals above the threshold level for all three replicates of 10 target copies. For 50 target copies the time to positivity for CCPSDA was 20 minutes (8/8 positive), compared with 17 minutes for Heated LAMP (5/8 positive) and 0/8 for traditional LAMP (Table 4). For 25 copies CCPSDA detected 5/8 replicates, while traditional and Heated LAMP both detected 0/8 replicates. For 10 copies CCPSDA detected 3/8 replicates while traditional LAMP detected 0/8 replicates and Heated LAMP detected 2/8 replicates.

TABLE 3
Comparison of amplification results for traditional
LAMP, Heated LAMP and CCPSDA
Time to positivity (minutes)

| | 100 copies | 50 copies | 25 copies | 10 copies |
|---|---|---|---|---|
| LAMP | 17.1^{a} | — | — | — |
| Heated | 13.1^{b} | 17.1^{d} | ND | 20^{g} |
| LAMP | | | | |
| CCPSDA | 15.6^{c} | 20^{e} | 16.6^{f} | 14.6^{h} |

*Time to positivity is expressed at the number of minutes to cross the detection threshold.
^{a}1/8 replicates were positive.
^{b}Mean of 8 replicates for 100 copies.
^{c}Mean of 8/8 replicates.
^{d}Mean of 5 replicates for 50 copies.
^{e}Mean of 8/8 replicates.
^{f}Mean of 5/8 replicates.
^{g}Mean of two replicates.
^{h}Mean of 3/8 replicates.
ND, not done.

TABLE 4
Amplification results for traditional
LAMP, Heated LAMP and CCPSDA

Number positive/Number tested for
various target copy numbers

| | 100 copies | 50 copies | 25 copies | 10 copies |
|---|---|---|---|---|
| LAMP | 1/8 | 0/8 | 0/8 | 0/8 |
| Heated | 1/8 | 5/8 | 0/8 | 2/8 |
| LAMP | | | | |
| CCPSDA | 8/8 | 8/8 | 5/8 | 3/8 |

### Example 3—CCPSDA Amplification Increases the Time for Non-Specific Products of Amplification to Reach Threshold Amplification Levels Compared to LAMP

The following example demonstrates the improved specificity of CCPSDA compared with LAMP.

CCPSDA and LAMP assays were performed using 10⁴ copies of human beta-actin gene target. CCPSDA reactions were 25 μL performed at 63° C. and consisted of using 1×AMP Buffer II.

LAMP reactions were performed at 63° C. using 1×AMP Buffer II which includes: 20 mM Tris-HCl, 10 mM (NH₄)₂SO₄, 150 mM KCl, 2 mM MgSO₄, 0.1% Tween® 20, pH 8.8 @ 25° C. for 1 hour either immediately or with indicated components incubated for 2 hours at 25° C. Reactions were performed in 25 μL volumes and consisted of 8 U Bst 3.0 DNA polymerase (New England Biolabs, Ipswich, Mass.), 20 ng/5 μL, human genomic DNA, and F3 and B3 primers 0.2 μM, LF and LB primers 0.4 μM F1P and B1P primers 1.6 μM, dNPTs 1.4 mM, Eva green dye.

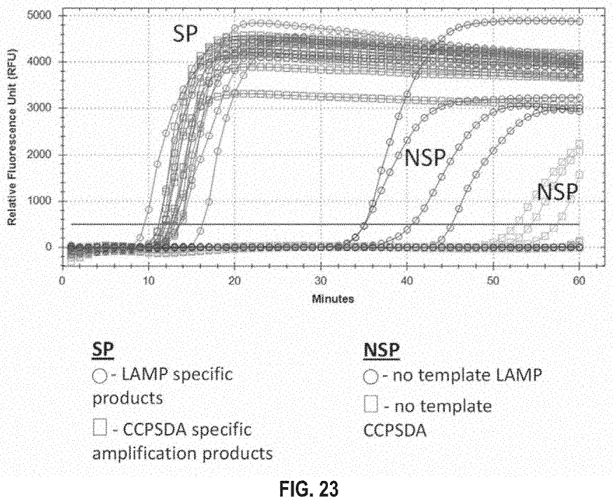

The time for non-specific products of amplification to reach threshold amplification levels was 34 minutes for LAMP compared to 52 minutes for CCPSDA as shown in FIG. 23.

### Example 4—CCPSDA Works with Only Two Co-Operative Primers

This example demonstrates that CCPSDA can work with only two CCP primers.

CCPSDA assays were performed in replicates of three using 10⁴ copies of Beta-actin gene target. CCPSDA reactions of 25 μL with two CCP primers alone or with two CCP primers and two loop primers together were performed at 63° C. and consisted of 1×AMP Buffer II. The concentrations of human Beta-actin CCP primers (Table 1) were F-CCP and R-CCP primers, 0.8 μM; LF and LB, 0.4 μM.

For CCPSDA amplification using CCP primers, the primer mix and template were heated to 94° C. for 4 mins, kept at 66° C. for a few minutes and cooled to room temperature just prior to addition to the reaction mixture. The primer/template mix was added to the reaction mix containing dNTP, Eva green, Bst 3.0, RNase H2 and amplified for 30 minutes at 63° C. on the BioRad CFX96.

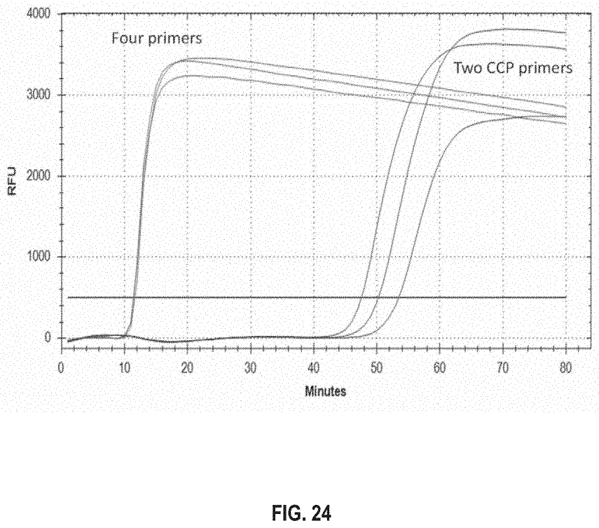

The results are shown in FIG. 24. CCPSDA with four primers, two CCP and two loop primers, crossed the amplification threshold at 10.5 minutes while the reaction with only two CCP primers was slower but crossed the threshold between 48 and 52 minutes.

Although preferred embodiments of the invention have been described herein, it will be understood by those skilled in the art that variations may be made thereto without departing from the spirit of the invention or the scope of the appended claims. All documents disclosed herein, including those in the following reference list, are incorporated by reference.

For example, the present invention contemplates that any of the features shown in any of the embodiments described herein, may be incorporated with any of the features shown in any of the other embodiments described herein, and still fall within the scope of the present invention.

### REFERENCES

1. Walker, G. T. et al. (1992) Strand displacement amplification an isothermal in vitro DNA amplification technique. Nucleic Acids Res 20:1691-1696.
2. Walker, G. T. et al. (1992). Isothermal in vitro amplification of DNA by a restriction enzyme/DNA polymerase system. Proc Natl Acad Sci USA 89:392-396.
3. Little, M. C. et al. (1999). Strand displacement amplification and homogeneous real-time detection incorporated in a second generation DNA probe system, BDProbe TecET. Clin. Chemistry 45:6 777-784.
4. Fire A, Xu S-Q. Rolling replication of short DNA circles. Proc. Natl. Acad. Sci. USA, 92 (1995) 4641 4645.
5. Liu D, Daubendiek S L, Zillman M A, Ryan K, Kool E T. Rolling circle DNA synthesis: small circular oligonucleotides as efficient templates for DNA polymerases. J Am Chem Soc. 1996; 118(7):1587-94.
6. Lizardi P, Huang X, Zhu Z, Bray-Ward Z, Thomas D, et al. Mutation detection and single molecule counting using isothermal rolling-circle amplification. Nature genetics. 19 (1998) 225-232.
7. Notomi T, Okayama H, Masubuchi H, Yonekawa T, Watanabe K et al. Loop-mediated isothermal amplification of DNA. Nucleic Acid Res. 28 (2000) e63.
8. Wahed A, Patel P, Heidenreich D, Hufert F, Weidmann M. Reverse transcription recombinase polymerase amplification assay for the detection of middle East respiratory syndrome coronavirus. PLoS Current. 5 (2013).
9. Vincent M, Xu Y, Kong, H. Helicase-dependent isothermal DNA amplification. EMBO reports. 5 (2004) 795-800.
10. Hall M, Wharam S, Weston A, Cardy D, Wilson W Use of signal mediated amplification of RNA technology (SMART) to detect marine cyanophage DNA. BioTechniques. 32 (2002) 604-611.
11. Satterfield B C. Cooperative Primers. 2.5 Million-fold improvement in the reduction of nonspecific amplification. Journal of Molecular Diagnostics (2014) 16(2):163-173.
12. Dobosy J R, Rose S D, Beltz K R, Rupp S M, Powers K M, Behlke M A, Walder J A. RNase H-dependent PCR (rhPCR): Improved specificity and single nucleotide polymorphism detection using blocked cleavable primers. BMC Biotechnology (2011) 11:80.
13. Van Ness. J, et al. PNAS 2003 100 (8): 4504-4509; Tan, E., et al. Anal. Chem. 2005, 77:7984-7992; Lizard, P., et al. Nature Biotech 1998, 6:1197-1202.
14. Wang J, Liu L, Wang J, Sun X, Yuan W. Recombinase Polymerase Amplification Assay-A Simple, Fast and Cost-Effective Alternative to Real Time PCR for Specific Detection of Feline Herpesvirus-1. PLoS One. 2017 Jan. 3; 12(1):e0166903. doi: 10.1371/journal.pone.0166903. eCollection 2017.

1. A target-specific co-operative primer for amplifying a target polynucleotide region of a nucleic acid molecule, the primer comprising:
a 3′ to 5′ bumper sequence segment,
a 5′ to 3′ inner primer sequence segment, the inner primer sequence segment comprising a capture sequence at the 3′ end of the inner primer sequence segment; and
a cleavage site located between the bumper sequence segment and the capture sequence segment, connecting the 5′ end of the bumper sequence segment to the 5′ end of the inner primer sequence segment;
wherein the cleavage site comprises one or more ribonucleotides that are cleavable by a RNase H enzyme.

2. (canceled)

3. (canceled)

4. The primer according to claim 1, wherein the cleavage site comprises a single ribonucleotide.

5. The primer according to claim 1, wherein the capture sequence segment has a higher melting temperature (Tm) than the bumper sequence segment.

6. The primer according to claim 5, wherein the Tm of the capture sequence segment is about 2° C. to 7° C. higher than the Tm of the bumper sequence segment.

7. The primer according to claim 6, wherein the Tm of the capture sequence segment is about 5° C. to 7° C. higher than the Tm of the bumper sequence segment.

8. The primer according to claim 1, wherein the bumper sequence segment anneals to the target polynucleotide region upstream of where the capture sequence segment anneals to the target polynucleotide region.

9. A kit for amplifying a target polynucleotide region of a nucleic acid molecule comprising, in one or more containers, at least two target-specific co-operative primers according to claim 1, a thermostable polymerase, a ribonuclease (RNase) enzyme and a buffer; wherein the at least two target-specific co-operative primers comprise:
(a) a first primer that anneals to a first region of the target polynucleotide region; and
(b) a second primer that anneals to a region of an extension product of the first primer.

10. (canceled)

11. The kit of claim 9, wherein the nucleic acid molecule is a double stranded DNA, and wherein the second primer anneals to a second region of the target polynucleotide region on a strand complementary to the first region.

12. The kit according to claim 9, wherein nucleic acid molecule is a double stranded DNA, and wherein the at least two target-specific co-operative primers comprise:
(a) the first primer that anneals to a first region of the target polynucleotide region;
(b) the second primer that anneals to a second region of the target polynucleotide region on the complementary strand;
(c) a third primer that anneals to a third region of the target polynucleotide region; and
(d) a fourth primer that anneals to a fourth region of the target polynucleotide region on the complementary strand.

13. The kit according to claim 9, further comprising two loop primers.

14. (canceled)

15. (canceled)

16. (canceled)

17. The kit according to claim 9, wherein the buffer has a pH in the range of pH 6-pH 9, and comprises a monovalent salt having a concentration in the range of 0-500 mM, and a divalent metal cation having a concentration of 0.5 mM-10 mM and optionally a stabilizing agent selected from the group consisting of BSA, glycerol, a detergent and mixtures thereof.

18. The kit according to claim 9, wherein the thermophilic polymerase has strand displacement activity and is active at temperatures greater than about 50° C.

19. The kit according to claim 9, wherein the buffer further contains a single stranded binding protein (SSB) in the range of 0.5 ug to 2 ug per reaction.

20. (canceled)

21. The kit according to claim 9, wherein the ribonuclease enzyme is RNase H2 enzyme.

22. The kit according to claim 9, further comprising a base repair enzyme.

23. (canceled)

24. A method of amplifying a target polynucleotide region of a nucleic acid molecule, comprising:
contacting the nucleic acid molecule with:
at least two target-specific co-operative primers according to claim 1, and a thermostable polymerase;
under a condition that promotes strand displacement amplification; and
cleaving the cleavage sites using a RNase H enzyme.

25. (canceled)

26. The method according claim 24, wherein the at least two target-specific co-operative primers comprise:
(a) a first primer that anneals to a first region of the target polynucleotide region; and
(b) a second primer that anneals to a region of the extension product of the first primer.

27. The method according to claim 24, wherein the at least two target-specific co-operative primers comprise:
(a) a first primer that anneals to a first region of the target polynucleotide region;
(b) a second primer that anneals to a second region of the target polynucleotide region on the complementary strand;
(c) a third primer that anneals to a third region of the target polynucleotide region; and
(d) a fourth primer that anneals to a fourth region of the target polynucleotide region on the complementary strand.

28. The method according to claim 24, further comprising contacting the nucleic acid molecule with two loop primers.

29. (canceled)

30. The method according to claim 24, further comprising containing the nucleic acid molecule with a single stranded binding protein (SSB), comprising:
(a) combining the single stranded binding protein (SSB) with the thermostable polymerase, the at least two primers and the nucleic acid molecule in a reaction buffer at a first temperature; and
(b) immediately or after a lag time at a temperature above 4° C. but below 70° C., performing an isothermal strand displacement amplification reaction at a second temperature, wherein the increase is determined with respect to the same mixture without the SBB.

31. The method according to claim 24, comprising performing PCR, qPCR, HDA, LAMP, RPA, TMA, NASBA, SPIA, SMART, Q-Beta replicase, or RCA.

32. The method according to claim 24, further comprising isolating the amplified target polynucleotide region, and
detecting the amplified target polynucleotide region using a fluorescent probe; a DNA binding dye; a PNA or BNA probe and a dye that recognizes PNA/BNA-DNA complexes, or a methylene blue dye for cyclic voltammetry.

33. (canceled)

34. The kit according to claim 13, comprising:
(a) a first primer comprising SEQ ID No: 1;
(b) a second primer comprising SEQ ID No: 2;
(c) a first loop primer comprising SEQ ID No: 3; and
(d) a second loop primer comprising SEQ ID No: 4.

35. The kit according to claim 13, comprising:
(a) a first primer comprising SEQ ID No: 5;
(b) a second primer comprising SEQ ID No: 6;
(c) a first loop primer comprising SEQ ID No: 7; and
(d) a second loop primer comprising SEQ ID No: 8.

36. The kit according to claim 13, comprising:
(a) a first primer comprising SEQ ID No: 9;
(b) a second primer comprising SEQ ID No: 10;
(c) a first loop primer comprising SEQ ID No: 11; and
(d) a second loop primer comprising SEQ ID No: 12.

37. The kit according to claim 9, further comprising a RNase inhibitor.

38. (canceled)


SEQUENCE LISTING (16 sequences)

SEQ ID NO: 1 - DNA, 70 bp, Artificial Sequence
  Primer
accccatgaagtcccactgctcctcgggagccacacgcagctcattgtagagcacggcatcgtcaccaac

SEQ ID NO: 2 - DNA, 68 bp, Artificial Sequence
  Primer
gcaacgataggtccgacaaggcccccctgaaccccaaggccaacccatggctggggtgttgaaggtct

SEQ ID NO: 3 - DNA, 22 bp, Artificial Sequence
  Primer
agattttctccatgtcgtccca

SEQ ID NO: 4 - DNA, 23 bp, Artificial Sequence
  Primer
cgagaagatgacccagatcatgt

SEQ ID NO: 5 - DNA, 73 bp, Artificial Sequence
  Primer
tcccgtaaaacctatttcgcatgacacctccttggccccatggaacgttgaaatggggacccgaacaacatgg

SEQ ID NO: 6 - DNA, 74 bp, Artificial Sequence
  Primer
agccagatcaaacacggtgactaagctattcaactggtgcacttgcaaggcttctgtggtcactgttcccatcc

SEQ ID NO: 7 - DNA, 25 bp, Artificial Sequence
  Primer
tgagcttcttgtatagtttaactgc

SEQ ID NO: 8 - DNA, 22 bp, Artificial Sequence
  Primer
tgcatgggcctcatatacaaca

SEQ ID NO: 9 - DNA, 71 bp, Artificial Sequence
  Primer
tactccgggtacgttgacctgtgctgggaatcagcaatctgctcacacaataggatgggggctgtaaccac

SEQ ID NO: 10 - DNA, 71 bp, Artificial Sequence
  Primer
atacctcgtttaccgacctaggtctcataggcagatggtggcaacacttagctgtagtgctggccaaaacc

SEQ ID NO: 11 - DNA, 21 bp, Artificial Sequence
  Primer
aatctgctcacatgttgcaca

SEQ ID NO: 12 - DNA, 25 bp, Artificial Sequence
  Primer
cattaataaaacatgagaacagaat

SEQ ID NO: 13 - DNA, 40 bp, Artificial Sequence
  Primer
gagccacacgcagctcattgtacacggcatcgtcaccaac

SEQ ID NO: 14 - DNA, 38 bp, Artificial Sequence
  Primer
ctgaaccccaaggccaaccggctggggtgttgaaggtc

SEQ ID NO: 15 - DNA, 19 bp, Artificial Sequence
  Primer
ccctgaagtaccccatcga

SEQ ID NO: 16 - DNA, 19 bp, Artificial Sequence
  Primer
acagcctggatagcaacgt